(19) United States
(12) Patent Application Publication
(10) Pub. No.: US 2005/0085554 A1
(43) Pub. Date: Apr. 21, 2005

(54) Methods of treating disease through the administration of a manzamine analog or derivative

(76) Inventors: Mark T. Hamann, Oxford, MS (US); Karumanchi Venkateswara Rao, University, MS (US); Jiangnan Peng, University, MS (US)

Correspondence Address: GREENBERG TRAURIG, LLP, MET LIFE BUILDING, 200 PARK AVENUE, NEW YORK, NY, 10166, US

(21) Appl. No.: 10/878,702

(22) Filed: Jun. 28, 2004

Related U.S. Application Data
(60) Provisional application No. 60/483,380, filed on Jun. 26, 2003.

(51) Int. Cl. A61K 31/47

(52) U.S. Cl. 514/649; 514/310

(57) ABSTRACT

A method of treating cancer, inflammatory disease or an infectious disease or condition in a subject in need of such treatment is disclosed. The method comprises administering to a subject an effective amount of a manzamine, or a rationally modified manzamine derivative or analog or an optical isomer or racemate or tautomer thereof or a pharmaceutically acceptable salt or prodrug thereof generated through optimized fermentation of a Micromonospora sp., extraction from sponges and then modified through semisynthesis.

### CROSS-REFERENCE TO RELATED APPLICATION

This application claims priority from U.S. Provisional No. 60/483,380, filed Jun. 26, 2003, which is incorporated by reference herein.

### UNITED STATES GOVERNMENT FINANCIAL SUPPORT

This research is supported in part by grants from the National Institutes of Health of the United States of America R01AI36596 and 5KO2AI01502. The Government of the United States may have certain rights in this invention.

### FIELD OF THE INVENTION

The present invention relates to the use of group of sponge-derived alkaloids known as or closely related to the “manzamines” as well as rationally prepared and natural derivatives and analogs thereof in the treatment of infectious diseases, cancer and inflammatory diseases. Manzamines are defined as a class of alkaloids isolated from the Phylum Porifera and containing a sophisticated array of aromatic and aliphatic rings. These metabolites are further divided into two groups. Those which contain a betacarboline or betacarboline related moieties attached to another functional system as in the manadomanzamines and papuamines and those metabolites in which the aliphatic alkaloid polycyclic system found in manzamine A contains functionality other than a betacarboline system as in ircinal and ircinol. See, Jinfeng Hu, Russell Hill, Michelle Kelly and Mark T. Hamann*. 2003. The Marine β-carboline Containing Alkaloids called the Manzamines for the series entitled: “The Alkaloids: Biology and Chemistry”. 205-283. Published by Elsevier. It has been found that manzamines as well as derivatives and analogs thereof are useful as anticancer and antiinflammatory agents; as antiparasitics, antimicrobials/bacteriology, antivirals and against fungal infections (mycology). Three new natural manzamine derivatives are also disclosed as well as new semisynthetic derivatives of the manzamine alkaloids.

### BACKGROUND OF THE INVENTION

Tuberculosis infects one-third of the world's population and, with malaria, ranks among the 12 leading causes for loss of Disability-Adjusted Life Years (“DALYs”). The rapid spread of drug-resistant strains of tuberculosis and malaria, coupled with the extremely limited numbers of drugs available to treat these diseases, has created an urgent need for novel therapeutic agents with new modes of action to counter these impeding threats.

Despite a significant reduction in mortality due to infectious diseases in the United States and Europe over the last century the last two decades have shown mortality increases that indicate the need for constant vigilance. See, Armstrong, G. L.; Conn, L. A.; Pinner, R. W., “Trends in Infectious Disease Mortality in the United States during the 20^{th} Century,” JAMA 1999, 281, 61-66.

The risk of malaria now exists in 100 countries and territories, with 92 of these facing the malignant and most dangerous form of the disease (Plasmodium falciparum). Over 45% of the world population lives in areas where malaria is endemic. Globally, there are 300-500 million clinical cases, with 1.5-2.7 million deaths associated with malaria annually. See, http://www.who.int/ctd/html/malaria.html (2000). Most of the deaths occur among children under five years of age. Despite the initial success of the World Health Organization's program to eradicate malaria globally during the 1950's and 1960's, it has become increasingly clear that these attempts have faltered due to increasing resistance of the malarial parasites to commonly used drugs and of the mosquito to insecticides. The estimated number of new infections has now reached their original levels, many of these being “malignant” malaria caused by, P. falciparum.

Although malaria is not considered an opportunistic infection in HIV-infected patients it has been shown that HIV positive individuals are more susceptible to P. falciparum and become more symptomatic. See, Verhoeff, F. H.; Brabin, B. J.; Hart, C. A.; Chimsuku, L.; Kazembe, P.; Broadhead, R. L., “Increased Prevalence of Malaria in HIV-Infected Pregnant Women and its Implications For Malaria Control,” Trop Med Int Health 1999, 4, 512. See also, French, N.; Gilks, C., “HIV and malaria, do they interact?,” Trans R Soc Trop Med Hyg 2000, 94, 23337. In addition, plasma viral loads have been shown to be higher in acutely infected malaria patients with HIV (see, Hoffman, I. F.; Jere; C. S.; Taylor, T. E., “The Effect of Plasmodium falciparum Malaria on HIV-1 RNA Blood Plasma Concentration, AIDS 1999, 13, 487-494) and malaria infections have been shown to induce virus expression in HIV transgenic mice. See, Freitag, C.; Chougnet, C.; Schito, M.; Near, K. A.; Shearer, G. M.; Li, C.; Langhorne, J.; Sher, A., “Malaria Infection Induces Virus Expression in Human Immunodeficiency Virus Transgenic Mice by CD4 T Cell-Dependent Immune Activation,” J. Infectious Diseases, 2001, 183, 1260-1268.

Tuberculosis (Mtb) remains today one of the most infectious diseases in the world. It is estimated that one-third of the world's population is infected by the tubercular organism, which claims the lives of 2-3 million people each year. In the large majority of those infected the infection remains latent, with only 10 percent ever developing active tuberculosis. The organism, however, is opportunistic and emerges to strike those with weakened immune systems, such as the elderly, AIDS patients, and people suffering from malnutrition. The infecting organism is a rod-shaped bacterium known as Mycobacterium tuberculosis.

Because relatively few drugs have been found satisfactory for the treatment of tuberculosis the occurrence of drug resistant tubercular bacilli looms with a frightening potential. Bacterial resistance to each of the presently available antituberculosis drugs has been observed, even with their combined use. The combined use of treatments involving rifampin and pyrazinamide has been shown to be potentially lethal. See, Morbidity and Mortality Weekly Reports 2001, 50, 289-291.

Resistance to current antituberculosis therapy is another threatening problem. Multi-drug-resistant strains of M. tuberculosis, resistant to as many as nine drugs, are 50-80% fatal even with intensive treatment. In the U.S., drug-resistant strains have been identified in seventeen states since 1989. Isoniazid resistance in the U.S. is present in 5.3% and secondary resistance in 19.4% of isolates, while the figures for rifampin are 0.6% and 3.2%, respectively. The resurgence of drug-resistant-tuberculosis has initiated a renewal of interest in a strategic search for new prototype leads. The oceans, with their unique and wide range of biodiversity, generating chemically diverse metabolites, emerge as an outstanding resource for new agents with anti-Mycobacterial activity. See, El Sayed, K. A.; Bartyzel, P.; Shen, X.; Perry, T. P.; Zjawiony, J. K.; and M. T. Hamann,” Marine Natural Products as Antituberculosis agents.” Tetrahedron 2000, 56, 949-953.

### BRIEF SUMMARY OF THE INVENTION

A class of sponge-derived alkaloids known as the “manzamines” show improved activity against malaria in mice over both chloroquine and artemisinin. The significant improvement in potency of the manzamines over the clinically used drugs in malaria-infected animals is due in part to an unprecedented half-life and significant oral availability. The manzamines have also shown significant activity in vivo against Mycobacterium tuberculosis and Toxoplasma gondii. The manzamines have also been shown to possess significant activity against inflammatory diseases, cancer, HIV-1 and other infectious diseases in vitro. Furthermore like many other classes of natural product derived drugs the manzamines have shown significant changes in bioactivity and selectivity which can be dramatically altered through synthetic modifications.

It has been found that the sponge-derived as well as sponge-associated microbial-derived manzamine related products including manadomanzamine and papuamine as well as rationally modified derivatives and analogs thereof are useful in the treatment of infectious diseases, cancer and inflammatory diseases based in part on modifications designed to improve metabolic stability, oral availability, reduced toxicity and improved biological activity. A number of these derivatives show enhanced selectivity and improved potency. The manzamines, manzamine derivatives, (including manadomanzamines and papuamines) and manzamine analogs are useful as anticancer agents, antiinflammatory, antiparasitics, antimicrobials (bacteriology) and antivirals and are suitable against opportunistic infections (mycology). The present invention also relates to the novel manadomanzamines and three new manzamine analogs identified from an Indo-Pacific sponge.

### DETAILED DISCLOSURE OF THE INVENTION

The manzamines are complex, polycyclic, marine-derived alkaloids first reported by Higa and coworkers in 1986 from the Okinawan sponge genus Haliclona. See, Sakai, R.; Higa, T.; Jefford, C. W.; Bernardinelli, G. “Manzamine A; An Antitumor Alkaloid From a Sponge,” J. Am. Chem. Soc. 1986, 108, 6404-6405. These compounds possess a fused and bridged tetra- or pentacyclic ring system that is attached to a β-carboline moiety. Since the first report of manzamine A, an additional forty manzamine-type alkaloids have been reported from a number of sponge genera (see, Hu, J. F.; Hamann, M. T.; Hill, R.; Kelly, M.; The Manzamine Alkaloids in “The Alkaloids: Biology and Chemistry” Edited by Geoffrey Cordell and Published by Elsevier June 2003).

The isolation of the manzamine alkaloids from a diversity of unrelated species provides strong evidence for a microbial origin for these metabolites including the manadomanzamines and papuamines. Manzamines exhibit a diverse range of bioactivities against cancer, inflammatory diseases, infectious organisms including antibacterial, antimycobacterial, cytotoxicity and the exciting and highly encouraging curative activity against malaria in animal models.

Manadomanzamines, new natural manzamine analogs, papuamines and manzamine semisynthetic products and compositions containing them can be administered via any suitable therapeutic method and technique presently or prospectively known to those skilled in the art. Further, the compounds for use in this invention have use as starting materials for the preparation of other useful drug products and compositions.

Skilled chemists having the benefit of the present disclosure of the structure of these manadomanzamines, manzamine analogs, papuamines and manzamine derivatives can readily use procedures to prepare the subject compounds from sponge/microbial extracts or through synthetic or biocatalytic transformations. In carrying out such operations, suitable filtration, chromatographic, crystallization and other purification techniques well known in the art may be used. These techniques may include, for example, reversed phase (RPLC), column, vacuum flash, medium pressure (MPLC) and high performance liquid chromatography (HPLC) with a suitable column such as silica gel, Sephadex LH-20, ammonia-treated silica gel, bonded phase RP-18, RP-8 and amino columns. Such columns are eluted with suitable solvents such as hexanes, ethyl acetate, acetone, methylene chloride, methanol, isopropanol, acetonitrile, water, trifluoroacetic acid (TFA) and various combinations thereof.

The dosage administered to a host will be dependent upon the identity of the cancer, inflammatory disease or infection, the type of host involved, its age, weight, health, kind of concurrent treatment, if any, frequency of treatment and therapeutic ration.

The compounds of the subject invention can be formulated according to known methods for preparing pharmaceutically useful compositions. Formulations are described in detail in a number of sources that are well known and readily available to those skilled in the art. For example, Remington's Pharmaceutical Sciences”, Mack Publishing Co., Easton, Pa., latest edition by E. W. Martin, describes formulations that can be used in connection with the subject invention. In general, the compositions of the subject invention will be formulated such that an effective amount of the bioactive compound(s) is combined with a suitable carrier in order to facilitate effective administration of the composition.

In accordance with the invention, pharmaceutical compositions comprising, as the active ingredient, an effective amount of one or more of the subject compounds and one or more non-toxic, pharmaceutically acceptable carriers or diluents, can be used by persons of ordinary skill in the art. In addition, the pharmaceutical composition can comprise one or more of the manadomanzamines, new manzamine analogs, papuamines and manzamine semisynthetic compounds as a first active ingredient together with a second or third active ingredient comprising an anticancer, antiinflammatory or antiinfective compound known in the art.

The most effective mode of administration and dosage regimen of the compounds as anticancer, antiinflammatory or antiinfective agents will depend upon the type of condition to be treated, the severity and course of that condition, previous therapy, the patient's health status and response to drug and the judgment of the treating physician. Manadomanzamine, manzamine analogs, papuamine and manzamine semisynthetic compositions may be administered to the patient at one time or over a series of treatments.

The present pharmaceutical formulations comprise a manadomanzamine, manzamine analog, papuamine or a manzamine derivative or analog or an optical isomer or racemate or tautomer thereof or a pharmaceutically acceptable salt thereof, optionally in a mixture with a pharmaceutically acceptable diluent or carrier. Further, the invention relates to the treatment of cancer, inflammatory or infectious diseases or conditions which comprise administering to a subject suffering from or susceptible to such a disease or condition, a therapeutically effective amount of a manadomanzamine, manzamine, papuamine a manzamine derivative or analog, or an optical isomer or racemate or tautomer thereof or a pharmaceutically acceptable salt thereof.

Any of the identified manadomanzamine, papuamines, manzamines and manzamine derivatives or analogs can be administered to an animal host, including a human patient, by itself, or in pharmaceutical compositions where it is mixed with suitable carriers or excipient(s) at doses therapeutically effective to treat or ameliorate a variety of cancers, inflammatory or infectious diseases. A therapeutically effective dose further refers to that amount of the compound sufficient to result in amelioration of symptoms associated with such disorders. Techniques for formulation and administration of the compounds of the instant application may be found in “Remington's Pharmaceutical Sciences”, Mack Publishing Co., Easton, Pa., latest edition.

Pharmaceutical compositions suitable for use in the present invention include compositions wherein the active ingredients are contained in an effective amount to achieve its intended purpose. More specifically, a therapeutically effective amount means an amount effective to prevent development of or to alleviate the existing symptoms of the subject being treated. Determination of the effective amounts is well within the capability of those skilled in the art, especially in light of the detailed disclosure provided herein.

For any compound used in the method of the invention, the therapeutically effective dose can be estimated initially from cell culture assays. For example, a dose can be formulated in animal models to achieve a circulating concentration range that includes the IC50 (the dose where 50% of the cells show the desired effects) as determined in cell culture. Such information can be used to more accurately determine useful doses in humans.

A therapeutically effective dose refers to that amount of the compound those results in amelioration of symptoms or a prolongation of survival in a patient. Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio between LD50 and ED50. Compounds that exhibit high therapeutic indices are preferred. The data obtained from these cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. The exact formulation, route of administration and dosage can be chosen by the individual physician in view of the patient's condition. (See e.g. Fingl et al., 1975, in “The Pharmacological Basis of Therapeutics”, Ch. 1 p1). Dosage amount and interval may be adjusted individually to provide plasma levels of the active moiety that are sufficient to maintain the desired effects.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries that facilitate processing of the active compounds into preparations that can be used pharmaceutically. Proper formulation is dependent upon the route of administration chosen.

For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated.

Pharmaceutical preparations for oral use can be obtained solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxy-methylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichloro-fluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g., by bolus injection or continuous infusion.

Formulations for injection may be presented in unit dosage form, e.g., in ampoules or in multidose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example) as an emulsion in an acceptable oil) or ion exchange resins, or as sparingly soluble derivatives, for example, as a sparingly soluble salt.

A pharmaceutical carrier for the hydrophobic compounds of the invention is a cosolvent system comprising benzyl alcohol, a nonpolar surfactant, a water-miscible organic polymer, and an aqueous phase. Naturally, the proportions of a co-solvent system may be varied considerably without destroying its solubility and toxicity characteristics. Furthermore, the identity of the co-solvent components may be varied.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various types of sustained-release materials have been established and are well known by those skilled in the art.

Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Most of the compounds of the invention may be provided as salts with pharmaceutically compatible counterions.

Pharmaceutically compatible salts may be formed with many acids, including but not limited to hydrochloric, sulfuric, acetic, lactic, tartaric, malic, succinic, etc. Salts tend to be more soluble in aqueous or other protonic solvents that are the corresponding free base forms.

Suitable routes of administration may, for example, include oral, rectal, transmucosal, transdermal, or intestinal administration; parenteral delivery, including intramuscular, subcutaneous, intramedullary injections, as well as intrathecal, direct intraventricular, intravenous, intraperitoneal, intranasal, or intraocular injections.

Alternately, one may administer the compound in a local rather than systemic manner, for example, via injection of the compound directly into an affected area, often in a depot or sustained release formulation.

Furthermore, one may administer the drug in a targeted drug delivery system, for example, in a liposome coated with an antibody specific for affected cells. The liposomes will be targeted to and taken up selectively by the cells.

The drugs may also be administered in a prodrug form in which a hydrolysable, oxidizable or reducible moiety has been formed at one or more reactive sites in the molecule. These include but are not limited to esters, sulphates, phosphates or any other group which can be readily metabolized to generate the active form of the drug.

The compounds of the subject invention can be used to treat a variety of cancers, inflammatory and infectious diseases in animals and humans including but not limited to: neurogenic inflammation, meningitis, septic shock, Down's syndrome, postischemic brain injury, HIV encephalopathy, Parkinson's disease, Alzheimer's disease, amyotrophic lateral sclerosis and multiple sclerosis.

The subject compounds and compositions can also be useful in the treatment of chronic pain, migraines, thermal-induced pain, such as sunburn, or other thermal and nociceptive pain, and chronic pain associated with arthritis. Uses can also include other inflammatory conditions that involve a neurogenic pain-producing component, e.g., certain metastatic carcinomas or inflammation of the blood vessels.

The compounds of the subject invention can also be used to treat a variety of skin conditions including, but not limited to, radiation irritation and burns (including UV and ionizing), chemical burns, rhinitis, thermal burns, reddening of the skin, and chemically induced lesions.

The compounds of the subject invention can also be used to treat allergic responses and/or promote wound healing. This can include the use of the compounds in aerosol form for the treatment of acute allergic reactions such as acute asthmatic attack and in the treatment of inflammation of the lung caused by chemical exposure.

The compounds of the subject invention can also be used to treat systemic anaphylactic reactions in animals and man.

Parasitic Diseases

Acanthamoeba castellani, Babesia microti, Cryptosporidium parvum, Entamoeba histolytica, Isospora belli, Leishmania chagasi, L. donovani, L. infantum, Naegleria fowleri, Plasmodium berghei, P. chabaudi, P. falciparum, P. malariae, P. ovale, P. vivax, P. yoelli, Pneumocystis carinii, Toxoplasma gondii, Trypanosoma brucei, T. brucei gambiense, T. brucei rhodesiense, T. cruzi, T. colubriformis, Nematodes [Intestinal (Ancylostoma duodenale, Ascaris lumbricoides, Necator americanus, Strongyloides stercoralis, Trichostrongylus spp., Trichuris trichiura)], [Tissue (Brugia malayi, Loa loa, Onchocerca volvulus, Trichinella spiralis, Wuchereria bancrofti)]

Antimicrobial/Bacteriology/Bioterrorism

Bacillus anthracis, B. subtilis, Borrelia burgdorferi, B. duttoni, B. hermsii, B. parkeri, B. recurrentis, B. turicatae, Bordatella parapertussis, B. pertussis, Branhamella catarrhalis, Chlamydia pneumoniae, C. psittaci, C. trachomatis, Clostridium botulism, C. difficile, C. novyi, C. perfringens, C. septicum, C. tetani, Corynebacterium diphtheriae, C. equi, C. haemolyticum, C. minutissimum, C. pseudodiphtheriticum, C. pseudotuberculosis, C. ulcerans, C. xerosis, C. jeikeium, Coxiella burnetii, Ehrlichia sennetsu, Eikenella corrodens, Enterobacter aerogenes, E. cloacae, Enterococcus coli, E. faecalis, Escherichia coli, Flavobacterium meningosepticum, Francisella tularensis, Haemophilus aegyptius, H. ducreyi, H. influenzae, H. parainfluenzae, H. para-aphrophilus, Hafnia alvei, Klebsiella pneumoniae, K. pneumoniae ssp. aerogenes, K. pneumoniae ssp. ozaenae, K. pneumoniae ssp. pneumoniae, K. pneumoniae ssp. rhinoscleromatis, Legionella pneumophila, L. pneumophila ssp. fraseri, L. pneumophila ssp. pneumophila, Leptospira biflexa, L. interrogans, Listeria monocytogenes, Moraxella phenylpyruvica, M. catarrhalis, M. lacunata, Morganella morganii, Mycobacterium avium-intracelluare, M. intracelluare, M. leprae, M. tuberculosis, Mycoplasma pneumoniae, Neisseria gonorrhoeae, N. meningitidis, Nocardia asteroides, Pasteurella pneumotropica, P. ureae, P. multocida, Peptostreptococcus spp., Proteus mirabilis, Pseudomonas aeruginosa, P. pseudomallei, Rickettsia prowazekii, R. rickettsii, R. sibirica, R. tsutsugamushi, R. typhi, Salmonella paratyphi, S. typhi, Serratia marcescens, Spirillum minus, Staphylococcus aureus, S. saprophyticus, S. epidermis, Streptobacillus moniliformis, Streptococcus agalactiae, S. anginosus, S. bovis, S. mitior, S. mutans, S. pneumoniae, S. pyogenes, S. salivanus, S. salivarius, S. sanguis, Treponema endemicum, T. pertenue, T. pallidum, Vibrio cholerae, Yersinia enterocolitica, Y. pseudotuberculosis, Y. pestis

Mycology

Aspergillus flavus, A. fumigatus, Blastomyces dermatitidis, Candida albicans, C. glabrata, C. guilliermondii, C. lusitaniae, C. parapsilosis, C. tropicalis, C. zephyr, Coccidioides immitis, Cryptococcus neoformans, Cryptosporidium spp., Fusarium ssp., Histoplasma capsulatum, H. duboisii, Mucor spp., Paracoccidioides brasiliensis, Penicillium spp., Pseudallescheria boydii, Rhizopus spp., Trichophyton rubrum, T. mentagrophytes, T. quinckeanum, Trichosporon beigelii.

Viral Diseases

Arbovirus (alphavirus, flavivirus, togavirus, bunyavirus), Arenavirus (Lassa fever virus), Coronavirus (HCV, 229E & OC43; HECV), Filovirus (Marburg, Ebola virus), Hepadnavirus (HBV, HDV, HEV, HCV), Lyssavirus (Rabies virus), Herpes virus (CMV, HCMVSCID-hu, MCMV, GPCMV, EBV, HSV-1, HSV-2, HHV6, HHV7, HHV8, VZV, Herpes B), Orbivirus, Papovavirus (HPV, polyomavirus), Poxvirus (monkey pox, small pox), Parvovirus (parvovirus B-19), Pircornavirus (coxsackie virus, echovirus, entervirus 70 & 71, HAV, poliovirus), Respiratory viruses (PIV, MV, HRV, AD), Orthomyxovirus (influenza A, B, C), Paramyxovirus (measles, mumps, NDV, parainfluenza virus, respiratory syncytial virus), Orthopoxvirus (Vaccinia, Cowpox), Retrovirus (HTLV-1, HTLV-2, HIV, HIV transmission inhibition, HIV-1, HIV-2).

Rearranged Manzamines known as Manadomanzamines

In one embodiment the subject invention pertains to the use of compounds having the following general structure: 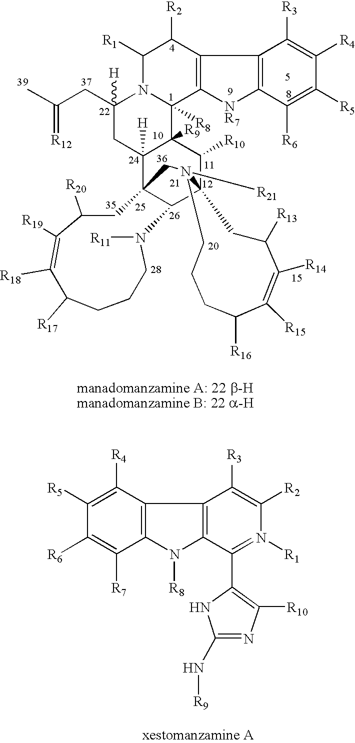
wherein R1-R21 are the same or different and are hydrogen, halogen, hydroxy, oxy, C₁-C₁₂-alkoxy, C₁-C₁₂-acyloxy, amide, amino, mono and dialkyl amino, aminal, thiol, C₁-C₁₂-alkylthiol, nitro, C₁-C₁₂-alkysulfonyl, aminosulfonyl, hydroxyl sulfonyl, C₁-C₁₂-acylamino, sulphate, C₁-C₁₂-alkyl, C₁-C₁₂-acyl or aryl groups including other drugs and natural products.

The frozen sponge can be easily collected from the Pacific or other Oceans by SCUBA in kilogram quantities. The sample can be extracted with acetone, ethanol, methanol or other suitable solvent and then chromatographed on silica gel, Al₂O₃, cellulose, or other bonded stationary phases to obtain manadomanzamines A and B, as well as the known compound xestomanzamine A (See, Kobayashi, M.; Chen, Y.; Aoki, S.; In, Y.; Ishida, T.; Kitagawa, I. Tetrahedron. 1995, 51, 3727-3736).

Manadomanzamine A (R10=OH; R12=O; All other R groups ═H) was isolated as a white powder and its molecular formula C₃₉H₅₄N₄O₂ was determined by HRESIMS 611.4348 [M+H] (calcd. 611.4319). The absence of the H-3 and H-4 aromatic signals in the ¹H NMR spectrum combined with the UV absorption at 282 nm suggested a tetrahydro-β-carboline. The stereochemistry of manadomanzamine A is also consistent with the corresponding stereochemistry of manzamine B, the proposed biogenetic precursor to the manadomanzamines. A positive CD Cotton effect at 226 nm was observed, indicating an R-configuration at C-1 of the tetrahydro-β-carboline ring. See, Sakai, R.; Kohmoto, S.; Higa, T.; Jefford, C. W.; Bernardinelli, G. Tetrahedron Lett. 1987, 28, 5493-5496. The absolute stereochemistry of the rest of the structure could readily be assigned relative to C-1.

Manadomanzamine B gives the same molecular formula as manadomanzamine A and its ¹H and ¹³C NMR data were comparable suggesting that it is diastereomeric in nature. The NOESY spectral data [H-1 (δ 4.13) to H-22 (δ 3.54) and H-24 (δ 1.98)] revealed an α configuration of H-22 for B, unlike H-22β for A. Manadomanzamine B has the same positive Cotton effect in the CD spectrum at 224 nm as A, suggesting the same R-configuration at C-1.

Manadomanzamines A and B represent a novel class of manzamine-type alkaloids with significant activity against both HIV-1 and Mtb.

Both manadomanzamines A and B exhibited significant activity against Mycobacterium tuberculosis (Mtb) with MIC values of 1.86 and 1.53 μg/mL (the MIC of the positive control, rifampin, is 0.16 μg/mL), suggesting manadomanzamines are a new class of anti-Mtb leads. Modification of manadomanzamines A and B through biotransformation and/or chemical synthesis is certain to yield new structures with improved biological activity. Manadomanzamines A, B, and xestomanzamine A are active against human immunodeficiency virus (HIV-1) with EC₅₀ values of 6.96, 16.5, and 11.2 μg/mL, respectively. Manadomanzamine A is active against human lung carcinoma A-549 and human colon carcinoma H-116 with IC₅₀ values of 2.5 and 5.0 μg/mL while manadomanzamine B is only active against H-116 with an IC₅₀ of 5.0 μg/mL. Manadomanzamines A, B, and xestomanzamine A did not show cytotoxicity against the normal Vero cell line (African Green Monkey kidney cells) at the tested concentration (4.76 μg/mL). Manadomanzamine B and xestomanzamine A are active against the fungus Cryptococcus neoformans with IC₅₀ values of 3.5 and 6.0 μg/mL. Manadomanzamine A was active against the fungus Candida albicans with an IC₅₀ of 20 μg/mL.

Bioactivity of Manadomanzamines A, B and Xestomanzamine A

| | | | | | Cytotoxicity | |
|---|---|---|---|---|---|---|
| | | | Antifungal IC₅₀ | | IC₅₀ | |
| | Anti-TB | Anti- | Candida | Crypto- | A- | H- |
| Compound | MIC | HIV-1 | albicans | coccus | 594 | 116 |
| Manadomanzam | 1.86 | 6.96 | 20 | NA | 2.5 | 5.0 |
| Manadomanzam | 1.53 | 16.5 | NA | 3.5 | >5 | 5.0 |
| Xestomanzami | 14.1 | 11.2 | NA | 6.0 | >5 | >5 |

A-594 = Human lung carcinoma;
H-116 = Human colon carcinoma;
“—” = Not Tested;
NA = Not Active.

The sponge can be collected easily from silty, sandy patch reefs between 10-20 m of depth, at Manado Bay, Indonesia. The sponge has a massive brown and reddish mass with upward digitate projections. This sample has a highly putrid odor with a fragile and easily torn texture. This sponge is a species of an undescribed Acanthostrongylophopra genus (Order Haplosclerida, Family Petrosiidae). A voucher specimen has been deposited at The Department of Pharmacognosy, University of Mississippi, and The National Institute of Water & Atmospheric Research Ltd., Auckland, New Zealand.

The sponge (4 kg, wet weight) can be extracted with acetone, ethanol, methanol or another suitable solvent. Fractions purified using silica gel was chromatographed on Al₂O₃ column eluted with hexane:acetone 8:2 then 7:3 to give manadomanzamine A (95 mg) and B (191 mg). A fraction (1.17 g) was dissolved in MeOH and pass through Sephadex LH-20 column (MeOH) to afford xestomanzamine A (140 mg).

Anti-HIV-1 activity was determined in PBM cells as described previously. See, Stock solutions (20 or 40 mM) of the compounds were prepared in sterile DMSO and then diluted to the desired concentration in growth medium. Cells were infected with the prototype HIV-1_{LAV} at a multiplicity of infection of 0.1. Details on the infection of cells and assessment of antiviral effects was described previously. See, Schinazi, R. F.; McMillan, A.; Cannon, D.; Mathis, R.; Lloyd, R. M.; Peck, A.; Sommadossi, J.-P.; Clair, M. S.; Wilson, J.; Furman, P. A.; Painter G.; Choi, W.-B.; Liotta, D. C. Antimicrob. Agents Chemother. 1992, 36, 2423-2431 and Schinazi, R. F.; Sommadossi, J.-P.; Saalmann, V.; Cannon, D. L.; Xie, M.-Y.; Hart, G. C.; Smith, G. A.; Hahn, E. F. Antimicrob. Agents Chemother. 1990, 34, 1061-1067.

Manadomanzamine A Physical Properties. White powder. HRESIMS 611.4348 [M+H] (calcd for C₃₉H₅₅N₄O₂ 611.4319); [α]_{D}−19° (c 0.11, MeOH); UV λₘₐₓ (nm) 282 (ε=7700); IR (film) ν 3372, 3002, 2919, 1707, 1468, 1354, 1164, 736; CD [θ]₂₀₁−74800, [θ]₂₀₈+40713, [θ]₂₂₆+44203, [θ]₂₇₁−11933 (c 4.50×10⁻⁵, MeOH); ¹H and ¹³C NMR data see Table below.

Manadomanzamine B Physical Properties. White powder. HRESIMS 611.4310 [M+H] (calcd for C₃₉H₅₅N₄O₂ 611.4319); [α]_{D}−18° (c 0.11, MeOH); UV λₘₐₓ (nm) 282 (ε=7.2×E+3); IR (film) ν 3387, 3001, 2917, 1711, 1460, 1355, 1162, 736; CD [θ]₂₀₀+66840, [θ]₂₀₅−45260, [θ]₂₂₄+52520, [θ]₂₆₉−12150 (c, 4.50×10⁻⁵, MeOH); ¹H and ¹³C NMR data see Table below.

TABLE
NMR Data of Manadomanzamine A (1) and B (2).

| | 1 | | | 2 | |
|---|---|---|---|---|---|
| | ¹H NMR | ¹³C NMR | ¹⁵N NMR | ¹H NMR | ¹³C NMR |
| 1 | 4.16 (d, 9.6) | 53.5 | | 4.13 (d, 9.3) | 60.1 |
| N-2^{a} | | | 46.3 | | |
| 3 | 3.25 (dt, 11.5, 6.0) | 49.3 | | 2.96 (m) | 40.4 |
| | 2.84 (m) | | | 2.90 (m) | |
| 4 | 2.80 (2H, m) | 23.0 | | 2.77 (2H, m) | 23.0 |
| 4a | | 108.7 | | | 108.3 |
| 5a | | 127.4 | | | 127.4 |
| 5 | 7.46 (d, 7.6) | 118.2 | | 7.45 (d, 7.6) | 118.2 |
| 6 | 7.04 (t, 7.6) | 119.0 | | 7.03 (t, 7.6) | 118.9 |
| 7 | 7.11 (t, 7.6) | 121.3 | | 7.10 (t, 7.6) | 121.3 |
| 8 | 7.31 (d, 8.0) | 111.3 | | 7.30 (d, 7.6) | 111.3 |
| 8a | | 136.1 | | | 136.1 |
| 9 | 9.17 (s) | | | 9.04 (s) | |
| N-9 | | | 123.8 | | |
| 9a | | 134.7 | | | 134.9 |
| 10 | 1.58 (m) | 43.0 | | 1.63 (m) | 40.4 |
| 11 | 3.51 (d, 2.7) | 74.5 | | 3.39 (d, 2.7) | 74.7 |
| 12 | | 64.7 | | | 64.6 |
| 13 | 2.47 (ddd, 15.1, | 31.0 | | 2.45 (m) | 31.4 |
| | 10.5, 4.0) | | | | |
| | 1.37 (m) | | | 1.20 (m) | |
| 14 | 2.63 (m) | 18.6 | | 2.54 (m) | 18.7 |
| | 1.80 (m) | | | 1.73 (m) | |
| 15 | 5.48 (m) | 130.0 | | 5.46 (m) | 130.1 |
| 16 | 5.29 (m) | 131.1 | | 5.30 (m) | 131.0 |
| 17 | 2.70 (m) | 25.0 | | 2.68 (m) | 25.0 |
| | 1.81 (m) | | | 1.82 (m) | |
| 18 | 1.15 (m) | 27.6 | | 1.16 (m) | 27.6 |
| | 1.77 (m) | | | 1.76 (m) | |
| 19 | 1.73 (m) | 27.0 | | 1.37 (m) | 27.0 |
| | 1.41 (m) | | | 1.74 (m) | |
| 20 | 2.55 (m) | 46.6 | | 2.52 (m) | 46.7 |
| | 2.12 (m) | | | 2.08 (m) | |
| N-21 | | | 36.9 | | |
| 22 | 3.75 (q, 5.6) | 57.9 | | 3.54 (m) | 58.6 |
| 23 | 1.44 (m) | 26.8 | | 1.49 (m) | 27.9 |
| | 1.78 (m) | | | 1.74 m) | |
| 24 | 2.11 (m) | 33.6 | | 1.98 (m) | 39.4 |
| 25 | | 45.2 | | | 45.3 |
| 26 | 2.93 (s) | 66.9 | | 2.95 (s) | 66.7 |
| N-27 | | | 32.6 | | |
| 28 | 3.09 (m) | 48.0 | | 3.10 (m) | 48.1 |
| | 2.72 (m) | | | 2.69 (m) | |
| 29 | 1.74 (2H, m) | 27.3 | | 1.65 (2H, m) | 27.4 |
| 30 | 1.47 (m) | 26.2 | | 1.46 (m) | 26.2 |
| | 1.86 (m) | | | 1.72 (m) | |
| 31 | 2.22 (m) | 24.3 | | 2.25 (m) | 24.3 |
| | 2.03 (m) | | | 1.98 (m) | |
| 32 | 5.53 (m) | 131.1 | | 5.55 (m) | 130.8 |
| 33 | 5.53 (m) | 130.8 | | 5.55 (m) | 130.6 |
| 34 | 2.05 (m) | 21.4 | | 2.26 (m) | 21.2 |
| | 2.24 (m) | | | 2.00 (m) | |
| 35 | 1.50 (m) | 33.3 | | 1.63 (m) | 33.1 |
| | 1.60 (m) | | | 1.44 (m) | |
| 36 | 2.88 (d, 10.0) | 57.5 | | 2.93 (d, 9.6) | 57.2 |
| | 2.39 (d, 10.0) | | | 2.28 (d, 9.6) | |
| 37 | 3.06 (dd, 16.6, 5.8) | 45.2 | | 2.89 (m) | 49.6 |
| | 2.63 (dd, 16.6, 8.6) | | | 2.54 (dd, 15.8, 7.6) | |
| 38 | | 208.6 | | | 207.4 |
| 39 | 2.17 (3H, s) | 30.9 | | 2.27 (s) | 30.6 |

^{a15}N NMR chemical shifts were measured using ¹H-¹⁵N HMBC spectra.
^{b}not observed.

Methods for Isolating 12,34-Oxamanzamine E, 6-Hydroxymanzamine E and 8-Hydroxymanzamine J. New Manzamines with applications against cancer, inflammatory and infectious diseases.

Animal material. The sponge sample which is extremely common was collected from near Manado, Indonesia in March 2002 and a voucher specimen is deposited in NIWA, Auckland, New Zealand and the Department of Pharmacognosy, University of Mississippi.

Extraction and Isolation. The sponge, Acanthostrongylophora sp. (MD02) was stored frozen until extracted. The lyophilized sponge (14 kg, wet weight) was crushed, homogenized and then extracted with acetone at room temperature. The extract was concentrated under reduced pressure and the resultant aqueous acetone extract was treated with chloroform. TLC analysis indicated that the extract contained manzamine A, together with various minor alkaloids as detected by Dragendorff reagent. The chloroform extract (120 g) was subjected to Si gel vacuum liquid chromatography and eluted in order, with hexane (100%), hexane-acetone (9:1, 3:1, 1:1), acetone (100%), chloroform-methanol (1:1) and methanol (100%). A total of ten major fractions were collected and the elution of metabolites was monitored by TLC.

12,34-Oxamanzamine E: pale yellow powder (MeOH). mp 152° C. (dec.), [α]_{D}²⁵+44.32 (c 0.6, CHCl₃). UV λₘₐₓ (log ε) (MeOH) 252 (3.82), 275 (3.65), 354 (3.41) nm. IR νₘₐₓ (CHCl₃) 3650, 3001-2818, 1716, 1620, 1592, 1533, 1452, 1267, 1144, 1052 cm⁻¹. ¹H-NMR and ¹³C-NMR (CDCl₃): Table below. HRMS m/z: (M+H)⁺563.3352.

8-Hydroxymanzamine J: pale yellow powder (CHCl₃). [α]_{D}²⁵+23.4 (c 0.2, CHCl₃). UV λₘₐₓ (log ε) (MeOH) 251 (3.62), 274 (3.68), 358 (3.39) nm. ¹H-NMR and ¹³C-NMR (CDCl₃): Table below.

6-Hydroxymanzamine E: yellow powder (CHCl₃). mp >198° C. dec. [α]_{D}²⁵+34.40 (c 0.2, CHCl₃): UV λₘₐₓ (log ε) (MeOH) 218 (3.64), 239 (3.63), 280 (3.25), 288 (3.09) and 346 (3.42) nm. IR νₘₐₓ (CHCl₃) 3629, 3388, 3001-2815, 1715, 1690, 1630, 1548, 1443, 1145, 1048 cm⁻¹. ¹H-NMR and ¹³C-NMR (MeOD): Table below. HRESIMS m/z: (M+H)⁺581.3477.

Table of ¹H and ¹³C NMR data for New Natural Manzamines

| | 12,34-Oxamanzamine | | | | | |
|---|---|---|---|---|---|---|
| C | A^{a} | | 8-Hydroxymanzamine J^{a} | | 6-Hydroxymanzamine E^{b} | |
| no | ¹³C | ¹H | ¹³C | ¹H | ¹³C | ¹H |
| 1 | 143.9 s | — | 143.8 s | — | 143.6 s | — |
| 3 | 138.8 d | 8.41 d, 5.2 | 137.5 d | 8.28 d, 5.3 | 136.5 d | 8.21 d, 5.3 |
| 4 | 114.2 d | 7.84 d, 5.2 | 113.8 d | 7.85 d, 5.3 | 113.4 d | 7.82 d, 5.3 |
| 4a | 129.9 s | — | 130.5 s | — | 130.2 s | — |
| 4b | 122.0 s | — | 121.9 s | — | 122.4 s | — |
| 5 | 121.8 d | 8.08 d, 7.8 | 109.7, d | 7.58 d, 7.7 | 105.5 d | 7.50 d, 2.3 |
| 6 | 120.4 d | 7.26 t, 8.0 | 121.2 s | 7.15, dd, 7.8 | 151.6 s | — |
| 7 | 128.8 d | 7.51 t, 7.4 | 113.4 d | 7.04 d, 7.8 | 118.7 d | 7.09 dd, 2.3, |
| 8 | 112.3 d | 7.55 d, 8.0 | 131.5 s | — | 113.3 d | 7.53 d, 8.8 |
| 8a | 140.8 s | — | 136.4 s | — | 134.8 s | — |
| 9a | 133.8 s | — | 128.4 s | — | 136.3 s | — |
| 10 | 142.8 s | — | 140.2 s | — | 139.7 s | — |
| 11 | 132.7 d | 6.24, s | 133.6 d | 6.35 s | 137.1 d | 6.28 s |
| 12 | 80.5 s | — | 60.3 s | — | 70.1 s | — |
| 13 | 40.3 t | 1.66 m, 2.35 m, | 40.8 t | 1.46 m, 1.96 [symbol] | 41.2 t | 1.80 m, 1.89 m |
| 14 | 23.1 t | 2.45 m, 2.85 m | 22.6 t | 2.01 m, 2.24 [symbol] | 30.1 t | 2.11 m, 2.21 m |
| 15 | 129.9 d | 5.34, br s | 129.5 d | 5.34 ddd, | 128.9 d | 5.65 dt, 4.7, |
| | | | | 7.2, 8.2,10. [symbol] | | |
| 16 | 129.8 d | 5.29, br s | 1321.4 d | 5.52 dt, 7.8, | 132.1 d | 5.52 q, 9.09 |
| 17 | 25.4 t | 1.73 m, 1.86 m, | 28.6 t | 2.89 m, 2.92m | 26.5 t | 1.67 m, 2.48 m |
| 18 | 30.0 t | 1.24 m, 1.52 m | 28.4 t | 1.54 m, 1.91 [symbol] | 27.4 t | 1.36 m, 1.41 m |
| 19 | 30.1 t | 1.38 m, 1.46 m, | 28.8 t | 1.54 m, 1.92 [symbol] | 23.1 t | 1.34 m, 1.72 m |
| 20 | 59.3 t | 2.28,m, | 53.3 t | 2.81 m, 2.92 [symbol] | 53.6 t | 2.37 m, 2.63 m |
| | | 2.71 m | | | | |
| 22 | 50.1 t | 2.04 m, 3.02 m, | 49.7 t | 1.94 m, 2.92 m | 50.2 t | 1.83 m, 2.72 m |
| 23 | 32.1 t | 2.59 m, 2.67 m | 32.3 t | 1.75 m, 1.98 [symbol] | 32.4 t | 1.45 m, 1.84 m |
| 24 | 46.3 d | 2.52 dd, | 44.8 d | 2.45 m | 40.3 d | 3.15 dd, 7.3, |
| | | 11.8, 5.5 | | | | |
| 25 | 38.6 s | — | 43.8 s | — | 45.7 s | — |
| 26 | 67.2 d | 4.36, s | 59.1 d | 3.75 s | 75.8 d | 3.65 s |
| 28 | 54.1 t | 2.84 m, | 58.9 t | 2.36 m, 3.55 [symbol] | 53.7 t | 2.66 m, 3.56 m |
| | | 3.38, m, | | | | |
| 29 | 23.3 t | 1.72 m, 1.76 m | 29.3 t | 1.69 m, 1.79 [symbol] | 32.5 t | 1.83 m, 1.90 m |
| 30 | 33.1 t | 1.64 m, 1.78 m | 29.4 t | 1.25 m, 1.78 [symbol] | 48.4 t | 2.65 m, 2.98 m |
| 31 | 206.2 s | — | 25.2 t | 1.73 m, 1.48 [symbol] | 216.6 s | — |
| 32 | 30.9 t | 2.75 m, 3.20 m | 129.8 d | 5.61 dt, 11.0, | 37.4 t | 1.80 m, 2.21 m |
| 33 | 30.5 t | 2.15 m, | 131.6 d | 5.36 br t, 8 | 26.5 t | 1.60 m, 1.79 m |
| | | 2.25 m | | | | |
| 34 | 101.8 s | — | 26.4 d | 2.19 m, 2.74 [symbol] | 65.5 d | 3.2 m |
| 35 | 47.4 t | 2.27 m, | 37.6 t | 1.44 m, 1.98 [symbol] | 47.4 t | 1.34 m, 1.42 m |
| | | 2.34 m | | | | |
| 36 | 66.3 t | 2.24 d, | 65.6 t | 2.04 m, 2.35 [symbol] | 67.1 t | 2.31 m, 2.65 m |
| | | 3.15 m, | | | | |

400 MHz for ¹H and 100 MHz for ¹³C NMR Carbon multiplicities were determined by DEPT experiments.
s = quaternary, d = methine, t = methylene carbons. Coupling constants (J) are in Hz.
^{a}NMR obtained in CDCl₃
^{b}NMR obtained in MeOD

Table of Bioactivity data of new manzamines

| | Activity in vitro | | | | | |
|---|---|---|---|---|---|---|
| | Mycobacterium | Plasmodium | P. falciparum | | | |
| | Tuberculosis | falciparum | (chlorine-resistant | | | Cytotoxicity |
| | (H37Rv) | (D6 clone) | W2 clone) | Leishmania donovani | | (Vero) |
| Compounds | MIC μg/mL | IC 50 ng/mL | IC 50 ng/mL | IC 50 μg/mL | IC 90 μg/mL | ng/ml |
| Ircinal A | 30.2 | NA | NA | | | NC |
| 12,34-Oxamanamine E | | | | | | |
| Manzamine J | 1.7 | 1300 | 750 | 25 | 45 | NC |
| Manzamine A | 1.53 | 4.5 | 8.0 | 0.9 | 1.8 | 1200 |
| (+)-8-Hydroxymanzamine A | 0.91 | 6.0 | 8.0 | 6.2 | 11 | 1100 |
| 6-Deoxymanzamine X | 1.77 | 1300 | 1400 | 3.2 | 7.5 | 4760 |
| Manzamine E | 3.76 | 3400 | 4760 | 3.8 | 6.8 | NC |
| Manzamine X | NT | 950 | 2000 | 5.7 | 11 | NC |
| Manzamine F | 2.56 | 780 | 1700 | 4.2 | 7 | NC |
| neo-Kauluamine | 2.0 | 1700 | 2800 | NT | NT | NC |
| 6-Hydroxymanzamine E | 0.4 | 780 | 870 | 2.5 | 4.3 | 4300 |
| 8-Hydroxymanzamine J | | | | | | |
| Manzamine A N-oxide | 3.9 | 11 | 13 | 1.1 | 3.8 | 420 |
| Ircinol A | 1.93 | 2400 | 3100 | NT | NT | NC |
| Rifampin | 0.5 | NT | NT | NT | NT | NT |
| Chloroquine | NT | 15.5 | 170 | NT | NT | NT |
| Artemisinin | NT | 10 | 6.3 | NT | NT | NT |
| Pentamidine | NT | NT | NT | 2.1 | 10 | NT |
| Amphotercin B | NT | NT | NT | 0.06 | 0.15 | NT |

NA = not active NT = not tested; NC = No Cytotoxicity (concentration: 4760 ng/ml)

Table of Antimicrobial and HIV data for Manzamines.

| | Assay | | | | |
|---|---|---|---|---|---|
| | Antibacterial | | Antifungal | | |
| | IC₅₀ (μg/mL) | | IC₅₀ (μg/mL) | | |
| | | | C. | M. | HIV |
| Compounds | S. aureus | MRS | neoformans | intracellulare | EC₅₀ (μM) |
| 12,34-Oxamanzamine E | NA | NA | NA | NA | 17.5 |
| neo-Kauluamine | NA | NA | 3.0 | 3.0 | NT |
| Manzamine J | | | | | NT |
| Manzamine A | 0.5 | 0.7 | 3.0 | 0.35 | 4.2 |
| Manzamine A-N-oxide | 0.5 | 0.35 | 1.0 | 0.2 | NT |
| 8-Hydroxymanzamine A | 0.9 | 4.0 | 3.0 | 1.0 | 0.59 |
| 6-Deoxymanzamine X | 1.5 | 1.5 | 2.0 | 6.25 | 1.6 |
| Manzamine E | NA | NA | NA | 12.5 | 13.1 |
| Manzamine F | NA | NA | 6.5 | 6.25 | 7.3 |
| 6-Hydroxymanzamine E | NT | NT | 5.5 | 3.5 | NT |
| 8-Hydroxymanzamine J | | | | | |
| Manzamime X | 1.0 | 0.75 | 3.0 | 1.5 | 2.3 |
| Ircinal A | NA | NA | NA | 50 | 6.8 |
| Ircinol A | NA | 30 | NA | 50 | 4.3 |
| Amphotericin B | NT | NT | 0.15 | NT | NT |
| Ciprofloxacin | 0.10 | 0.10 | NT | 0.25 | NT |

NA = not active NT = not tested

Methods for Isolating the Papuamines and Applications against Methicillin Resistant Staph; Mtb and other infectious diseases.

In one embodiment the subject invention pertains to the use of compounds having the following general structure: 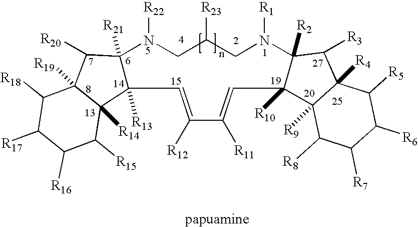
wherein R1-R23 are the same or different and are hydrogen, halogen, hydroxy, oxy, C₁-C₁₂-alkoxy, C₁-C₁₂-acyloxy, amide, amino, aminal, thiol, C₁-C₁₂-alkylthiol, nitro, C₁-C₁₂-alkysulfonyl, aminosulfonyl, hydroxyl sulfonyl, C₁-C₁₂-acylamino, sulphate, C₁-C₁₂-alkyl, C₁-C₁₂-acyl or aryl groups including other drugs and natural products and n is equal to 0-20. Also included are analogs generated through reduction or further oxidation of olefinic and aromatic functionality. Bioassay guided separation led to the isolation of the active alkaloid (−)-papuamine along with taurine, benzoethylamine, thymidine, 2′-deoxycytidine, 3-methl thymidine, uracil, thymine, and uridine. A combination of papuamine with these other primary metabolites shows improved activity and the drug maybe utilized in combination with these other compounds in order to enhance biological activity and deliverability. Papuamine was first isolated from a bright red encrusting sponge (Haliclona sp.) by Scheuer and co-workers in 1988. (see Baker, B. J.; Scheuer, P. J.; Shoolery, J. N. J. Am. Chem. Soc. 1988, 110, 965-966. Shortly thereafter, Faulkner, Clardy and co-workers reported haliclonadiamine, a diastereomer of papuamine, and a small amount of papuamine from a similar sponge collected in Palau. See, Fahy, E.; Molinski, T. F.; Harper, M. K.; Sullivan, B. W.; Faulkner, D. J. Tetrahedron, 1988, 29, 3427-3428.

Total synthesis of (+)- and (−)-papuamine was reported in 1994 and 1995 by two different groups. See, A. Barret, A. G. M.; Boys, M. L.; Boehm, T. L. J. Chem. Soc., Chem. Commun. 1994, 1881-1882. B. Borzilleri, R. M.; Weinreb, S. M.; Parvez, M. J. Am. Chem. Soc. 1994, 116, 9789-9790. C. Borzilleri, R. M.; Weinreb, S. M.; Parvez, M. J. Am. Chem. Soc. 1995, 117, 10905-10913.

The sponge Haliclona sp. was collected in March 2001 from Manado Bay, Indonesia using SCUBA. The methanol extract of the freeze-dried sponge was subjected to silica gel vacuum liquid chromatography, and then purified by C₁₈ and C₈ HPLC to yield papuamine and the other nine compounds.

Papuamine was obtained as a white powder and the molecular formula C₂₅H₄₀N₂ was determined by HRESIMS 369.3399 [M+H] (calcd. 369.3270). Its structure was elucidated by detailed analysis of 1D and 2D NMR data and comparison with the literature. See, Baker, B. J.; Scheuer, P. J.; Shoolery, J. N. J. Am. Chem. Soc. 1988, 110, 965-966. ¹H NMR (D₂O) δ 6.48 (2H, complex dd, J=7.2, 20.5 Hz, H-16, 17), 5.81 (2H, br dd, J=11.2, 11.2 Hz, H-15, 18), 3.48 (2H, dd, J=7.5, 15.5 Hz, H-6, 27), 3.06 (2H, m, 2H, H-2, 4), 2.64 (2H, q, J=9.0 Hz, H-14, 19), 2.39 (2H, m, H-7, 26), 1.95 (2H, br, H-3), 1.84 (2H, m, H-9, 24), 1.80 (2H, m, H-12, 21), 1.70 (4H, br, H-10, 11, 22, 23), 1.31 (2H, m, H-13, 20), 1.15-1.23 (6H, m, H-8, 10, 11, 22, 23, 25), 1.03 (br t, J=11 Hz, H-9, 24), 0.95 (m, H-12, 21). ¹³C NMR (D₂O) δ 134.3 (d, C-16, 17), 129.2 (d, C-15, 19), 61.5 (d, C-6, 27), 48.7 (d, C-13, 25), 48.1 (d, C-14, 19), 43.4 (d, C-8, 25), 45.7 (t, C-2, 4), 37.9 (t, C-7, 26), 31.1 (t, C-9, 23), 29.6 (t, C-12, 21), 26.0 (t, C-10, 11, 22, 23), 23.3 (t, C-3). Its optic rotation [α]_{D}−188 corresponded to (−)-papuamine. See, Borzilleri, R. M.; Weinreb, S. M.; Parvez, M. J. Am. Chem. Soc. 1994, 116, 9789-9790 and Borzilleri, R. M.; Weinreb, S. M.; Parvez, M. J. Am. Chem. Soc. 1995, 117, 10905-10913.

Papuamine exhibits significant activities against bacteria, fungi and protozoa. It showed selective activity against fungus Cryptococcus neoformans with IC₅₀/MIC of 0.35/0.63 μg/mL, which is comparable with the positive control amphotericin B. Papuamine exhibited a comparable activity with rifampin against Mycobacterium tuberculosis. Papuamine showed same activity against Staphylococcus aureus and its methicillin-resistant strain and could be utilized in the treatment of methicillin-resistant Staphylococcus infections. Papuamine is also active against malaria D6 and W2 clones of Plasmodium falciparum. The significant antimicrobial activities indicated papuamine is a significant lead for treatment of AIDS-OI's.

Antimicrobial Activities of Papuamine

| | Papuamine | | | | |
|---|---|---|---|---|---|
| | (μg/mL) | | Control (μg/mL) | | |
| | IC₅₀ | MIC | IC₅₀ | MIC | drug |
| Candida | 1.5 | 5.0 | 0.10 | 0.63 | amphotericin B |
| albicans | | | | | |
| Cryptococcus | 0.35 | 0.63 | 0.25 | 0.63 | amphotericin B |
| neoformans | | | | | |
| Staphylococcus | 2.5 | 5.0 | 0.10 | 0.63 | ciprofloxacin |
| aureus | | | | | |
| MRS | 2.5 | 5.0 | 0.10 | 0.31 | ciprofloxacin |
| Mycobacterium. | 4.0 | | 0.25 | 0.63 | ciprofloxacin |
| intracellulare | | | | | |
| Aspergillus | | 1.25^{a} | | 0.63^{a} | amphotericin B |
| fumigatus | | | | | |
| Plasmodium | 0.15 | | 0.01 | | chloroquine |
| falciparum (D6 | | | | | |
| clone) | | | | | |
| P. falciparum | 0.34 | | 0.14 | | chloroquine |
| (W2 clone) | | | | | |
| Mycobacterium | | 0.4 | | 0.24 | rifampin |
| tuberculosis | | | | | |

MIC: lowest concentration that allows no detectable growth.
MRS: methicillin-resistant Staphylococcus aureus.
^{a}the lowest concentration that significantly inhibits growth.

Methods for Preparing Papuamine Analogs

Due to the significant activity against AIDS-OI for the papuamines a synthesis of hundreds of analogs will further enhance the biological activity for this compound. A synthetic approach is most practical in this case due to the fact that the yield is modest from the sponge which is small and time consuming to collect. In addition this molecule is reasonably simple, making it a rational synthetic target. Using an efficient synthetic approach from the literature this molecule can easily be modified to prepare an unlimited number of analogs for optimization of the activity of papuamine against infectious diseases. See Barrett, A. G. M., M. L. Boys, T. L. Boehm. 1994. “Total Synthesis of (+)-Papuamine: Determination of the Absolute Stereochemistry of the Natural Product” Journal of Chem. Soc., Chem. Commun., 0, 1881-1882.

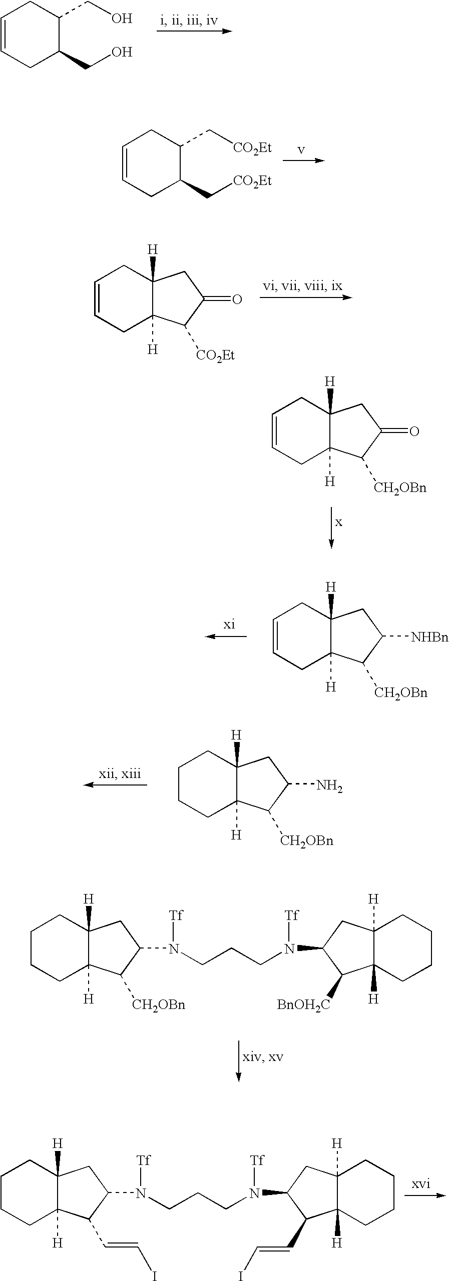

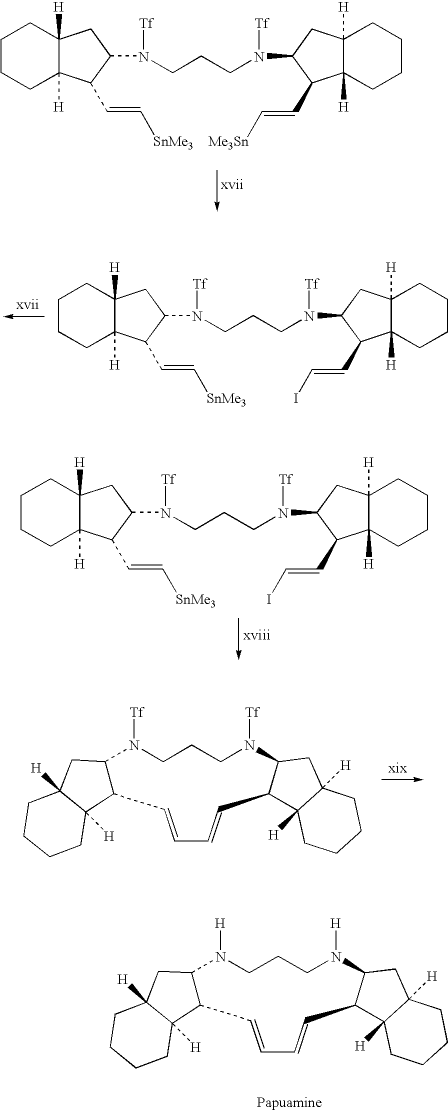

Figure Scheme for the Total Synthesis of Papuamine. i, TsCl, pyridine, 83%; ii, NaCN, EtOH, 86%; iii, KOH, H₂O, heat, 95%; iv, EtOH, H₂SO₄, 99%; V, NaH, THF, heat, 95%; vi, ethylene glycol, TsOH, Ph—H, molecular sieves, heat, 100%; vii, LiAlH₄, Et₂O, 99%; viii, NaH, BnBr, DMF, 92%; ix, 0.2 mol dm⁻³ HCl, THF 25-60° C., 97%; x, benzylamine, AcOH, NaBH(OAc)₃, THF, 85%; xi, NH₄O₂CH, 10% Pd/C, MeOH, heat, 86%; xii, (CF₃SO₂)₂O, Et₃N, CH₂Cl₂, −78° C., 97%; xiii, 1,3-dibromopropane, K₂CO₃, KI (cat), MeCN, heat, 90%; xiv, H₂, 10% Pd/C, EtOH, 95%; xv, (a) Swern oxidation, (b) CHI₃, CrCl₂, 1,4-dioxane/THF (6:1), 71%; xvi, hexamethylditin, Li₂CO₃, PdCl₂(PPh₃)₂, THF, 60° C., 51%; xvii, I2 (1 equiv.), Et2O, 44%; xviii, Pd(PPh₃)₄, (30 mol %), PhCH₃, 100° C., 39%; xix, LiAlH₄, Et₂O, heat, 42%.

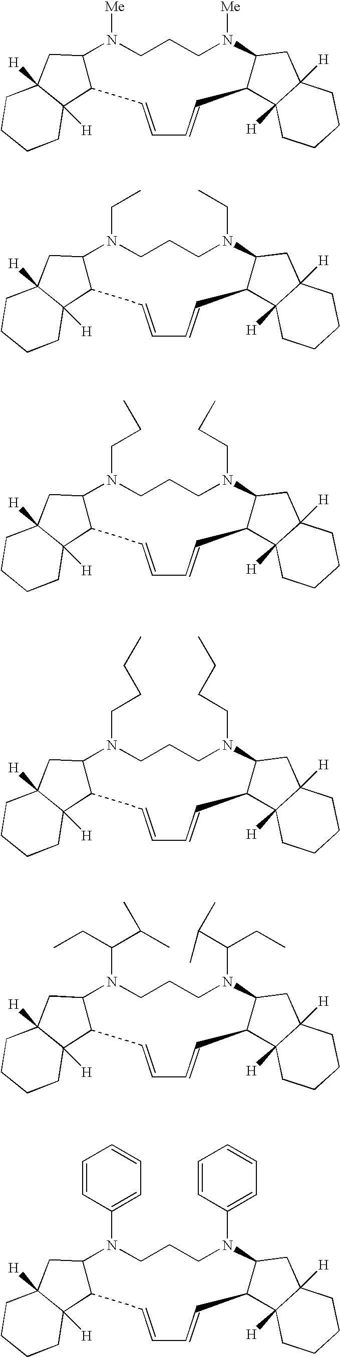

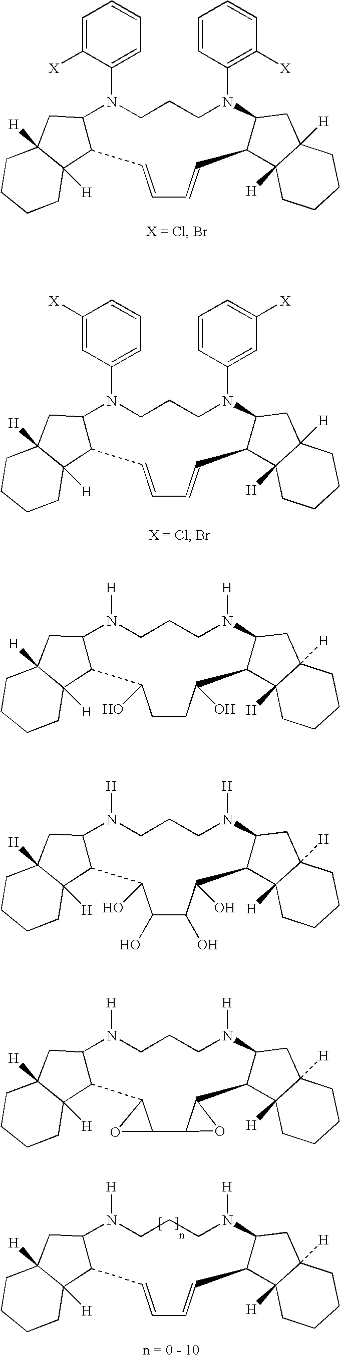

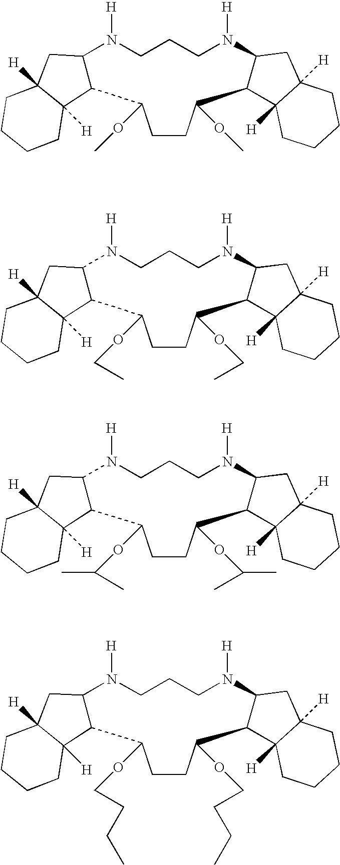

Examples of papuamine analogs that can be readily prepared using the published synthetic route to papuamine.

Manzamine Analogs

In one embodiment the subject invention pertains to the use of compounds having the following general structure: 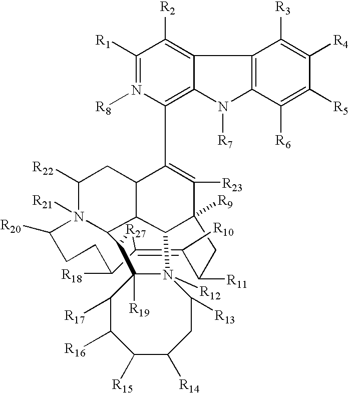
wherein R1-R23 are the same or different and are hydrogen, halogen, hydroxy, oxy, C₁-C₁₂-alkoxy, C₁-C₁₂-acyloxy, amide, lower mono or dialkyl amino, aminal, thiol, C₁-C₁₂-alkylthiol, nitro, C₁-C₁₂-alkysulfonyl, aminosulfonyl, hydroxyl sulfonyl, C₁-C₁₂-acylamino, sulphate, C₁-C₁₂-alkyl, C₁-C₁₂-acyl or aryl groups including other drugs and natural products. In addition reduction or oxidation of olefinic moieties is included as a subject of this invention as well as N-substituted analogs with the functionality mentioned previously.
Methods for Preparing Analogs of Manzamines E and F

Using the reaction scheme below alkaloids with improved 5 stability, kinetics and bioactivity can be prepared for use as treatments to control cancer, infectious and inflammatory diseases and R is any combination of hydrogen, alkyl, acyl, or aryl. 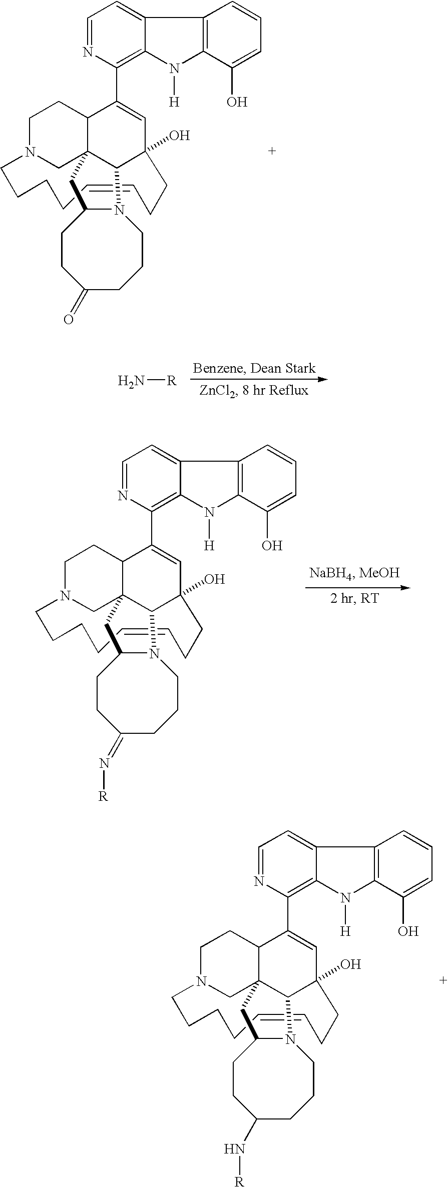
Methods for Preparing C-12 Manzamine Ether Analogs at the C-12 Hydroxy Group

Using the reaction scheme below ethers with improved metabolic stability and kinetics can be prepared for use as treatments to control cancer, infectious and inflammatory diseases and R is any combination of hydrogen, alkyl, acyl, aryl, sulphate, phosphate.

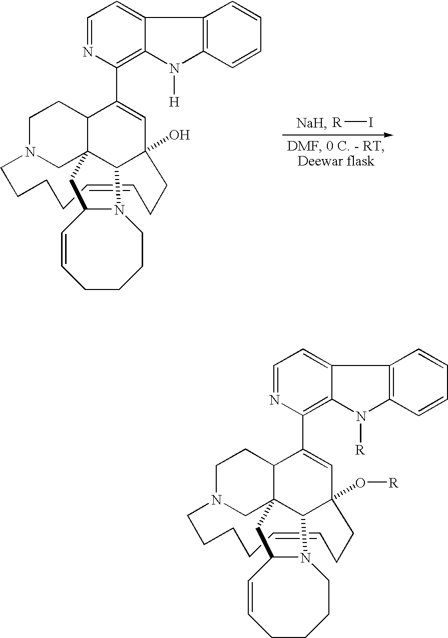

Table of Bioactivity data of Manzamine-A ether derivatives

| | Activity in vitro | | | | | |
|---|---|---|---|---|---|---|
| | | Plasmodium | P. falciparum | | | |
| | | falciparum | (chlorine-resistant | | | Cytotoxicity |
| | M. intracellulare | (D6 clone) | W2 clone) | Leishmania donovani | | (Vero) |
| Compounds | IC₅₀ (μg/mL) | IC 50 ng/mL | IC 50 ng/mL | IC 50 μg/mL | IC 90 μg/mL | ng/ml |
| 12-O-methylmanzamine-A | 2.0/5.0 | 1900 | 1000 | 6.5 | 16 | NC |
| 12-O-ethylmanzamine-A | | | | 2.8 | 8.2 | |
| 12-O-propylmanzamine-A | | | | 4.5 | 8.7 | |
| 12-O-isobutylmanzamine-A | | | | 0.98 | 2.6 | |
| 12-O-butylmanzamine-A | | | | 4.7 | 9.1 | |
| Manzamine A | 1.53 | 4.5 | 8.0 | 0.9 | 1.8 | 1200 |

Table of antimalarial and cytotoxicity data of 33-
aminomanzamine-F derivatives.

| | Activity in vitro | | |
|---|---|---|---|
| | Plasmodium | P. falciparum | Cyto- |
| | falciparum | (chlorine-resistant | toxicity |
| | (D6 clone) | W2 clone) | (Vero) |
| Compounds | IC 50 ng/mL | IC 50 ng/mL | μg/ml |
| 33-(2,5-dimethoxyaniline)- | 1700 | 2600 | NC |
| manzamine-F | | | |
| 33-(p-chlorobenzylamino)- | 680 | 1300 | NC |
| manzamine-F | | | |
| 33-phenethylamino- | NA | NA | NC |
| manzamine-F | | | |
| Manzamine-F | 780 | 1700 | NC |

NC = 4.7 μg/mL

Methods for Preparing Analogs of Ircinol or Ircinal.

In one embodiment the subject invention pertains to the use of compounds having the following general structure: 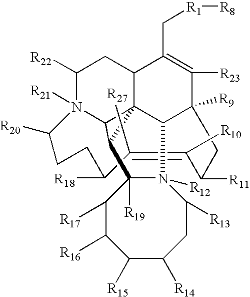
wherein R1-R23 are the same or different and are hydrogen, halogen, hydroxy, oxy, C₁-C₁₂-alkoxy, C₁-C₁₂-acyloxy, amide, lower mono or dialkyl amino, aminal, thiol, C1-C₁₂-alkylthiol, nitro, C₁-C₁₂-alkysulfonyl, aminosulfonyl, hydroxyl sulfonyl, C₁-C₁₂-acylamino, sulphate, C₁-C₁₂-alkyl, C₁-C₁₂-acyl or aryl groups including other drugs and natural products. In addition reduction or oxidation of olefinic moieties is included as a subject of this invention as well as N-substituted analogs with the functionality mentioned previously. The substituents R1-R8 are substitutes on a ring system derived from the OH ircinol or more important the aldehyde of ircinal. These ring systems can include aromatic, heteroaromatic, aliphatic mono or polycyclic rings ranging in ring size from C-3 to C-8 and ring numbers from 1-6 in addition to the functionality listed above.

Ircinol A (0.075 mol) was dissolved in dry methylene chloride (0.075 mol). To this solution, carboxylic acids (0.075 mol) and dimethyl aminopyridine (catalytic amount) were added and stirred for about 5 minutes followed by the addition of N,N′-dicyclohexylcarbodiimide (0.075 mol). The reaction was allowed to stir at room temperature and progress was monitored on silica gel on a PTLC aluminum card. All the reactions were stopped after 18 hours and the reaction mixture was filtered and evaporated.

| | | Yield |
|---|---|---|
| Entry | Product | (%) |
| 1a | 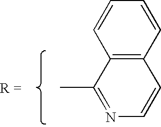 | 76 |
| 2a | 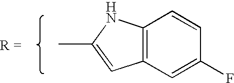 | 81 |
| 3a | 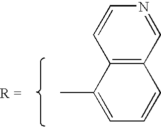 | 89 |
| 4a | 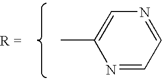 | 72 |
| 5a | 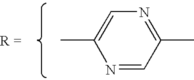 | 75 |
| 6a | 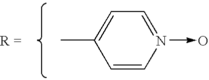 | 78 |
| 7a | 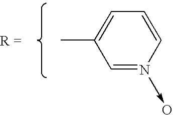 | 72 |
| 9a | 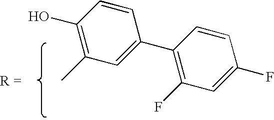 | 87 |
| 10a | 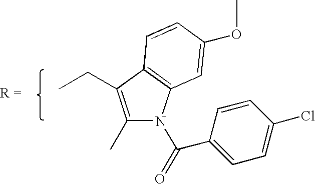 | 84 |
| 11a | 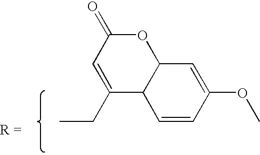 | 74 |
| 13a | 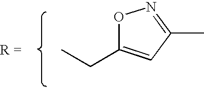 | 79 |
| 14a | 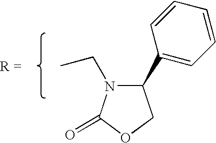 | 80 |
| 16a | 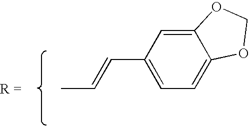 | 77 |
| 17a | 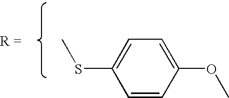 | 78 |
| 18a | 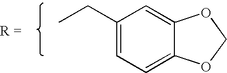 | 73 |
| 19a | 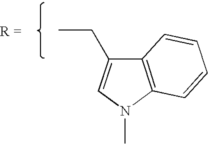 | 76 |
| 20a | 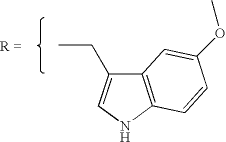 | 83 |
| 21a | 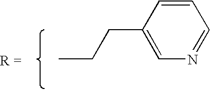 | 72 |
| 22a | 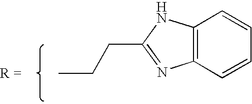 | 82 |
| 23a | 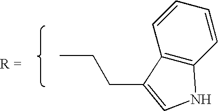 | 80 |

TABLE
Anti-HIV-1 Activity for Ircinol Semisynthetics

| | Anti-HIV activity | | Toxicity (IC₅₀-μM) in: | | |
|---|---|---|---|---|---|
| | in Humans ] | | IC₅₀-μM | IC₅₀-μM | IC₅₀-μM |
| Compound | EC₅₀ (μM) | EC₉₀ (μM) | PBM | CEM | Vero |
| 1a | 7.9 | 60.4 | 4.7 | 4.0 | 2.3 |
| 2a | 1.1 | 11.5 | 4.0 | 4.1 | 9.4 |
| 3a | 7.1 | 57.9 | 4.2 | 6.3 | 11.8 |
| 4a | 20.2 | >100 | 14.1 | 7.5 | 13.5 |
| 5a | 13.2 | 57.9 | 13.5 | 10.4 | 16.2 |
| 6a | 10.3 | 35.6 | 5.1 | 6.6 | 29.1 |
| 7a | 1.9 | 77.2 | 10.4 | 10.3 | <1.0 |
| 9a | | | | | |

TABLE
Anti-TB, Malarial, and Leishmania Data for Ircinol
Semisynthetics

| | Activity in vitro | | | | | |
|---|---|---|---|---|---|---|
| | | | P. falciparum | | | |
| | Mycobacter | Plasmodium | (chlorine- | Leishmania | | |
| | tuberculos | falciparum | resistant | donovani | | Cytotoxicity |
| | (H37Rv) | (D6 clone) | W2 clone) | IC₅₀ | IC₉₀ | (Vero) TC₅₀ |
| Compound: | MIC μg/mL | IC₅₀ ng/mL | IC₅₀ ng/mL | μg/mL | μg/r | (ng/ml) |
| 1a | 7.6 | 4700 | >4760 | 9.5 | 33 | NC |
| 2a | 1.7 | 2000 | 870 | 26 | 45 | NC |
| 3a | 6.4 | 3000 | NA | 8.5 | 29 | NC |
| 4a | 15.5 | 3000 | 3300 | 6.7 | 12 | NC |
| 5a | 14.4 | 3500 | 3400 | 6.7 | 12 | NC |
| 6a | 27.6 | 1800 | 400 | 7.5 | 12 | NC |
| 7a | 128.0 | NA | NA | 7.4 | 16 | NC |
| 9a | | | | | | |

Methods for obtaining the compounds are described in, for example, U.S. Pat. Nos. 4,895,852; 4,895,853; 4,895,854; 6,602,881 and International Patent Application number PCT/US00/07974; which are herein incorporated in their entirety by reference thereto.

The aliphatic portion of this alkaloid appears to be the key pharmacophore for antiMtb and antimalarial activity in vitro with the beta carboline also contributing to the activity in vivo. As a result the aldehyde of ircinal A may be utilized as a starting material to generate a tremendous number of bioactive derivatives (including but not limited to aliphatic, aromatic, heterocyclic systems originating from this functionality. These derivatives may be utilized for the treatment of infectious disease as well as cancer and inflammatory diseases as outlined in earlier patents which are included here in their entirety. The oxidation, reduction, formation of Schiff's bases, etc. at the aldehyde position is virtually endless. The primary focus however will be to utilize the aldehyde for the formation of additional heterocyclic moieties. See, Joule, J. A.; Mills, K.; Smith, G. F. “Heterocyclic Chemistry” Third Edition 1995 pp Chapman & Hall, New York. If, for some reason, a retro Pictet-Spengler condensation can not be made to work there will be sufficient quantities of ircinal A available by isolation from the sponges to study the chemistry at this position. A valuable aspect of this project is that through the molecular and microbiology proposed earlier a genetically engineered route to ircinal A is also conceivable. The goal of the semisynthesis will be to focus on the introduction of additional heteroaromatic functionality at the aldehyde position. A valuable result of this project will be additional synthetic methodologies using the Pictet-Spengler reaction for the formation of complex alkaloids semisynthetically.

In vitro Cytotoxicity to Mammalian Cells. Vero cells (ATCC #CCL 81) are obtained from the American Type Culture Collection. The Vero cells served as an indicator of general cytotoxicity. Vero cells are grown in RPMI 1640 medium (Sigma Chemical Co., St. Louis, Mo.) supplemented with glutamine, sodium bicarbonate and 10% fetal calf serum (Hyclone Laboratories) at 37° C. in a 5% CO₂ humidified atmosphere. The cells are allowed to grow for 48-72 h, at which time the cells are subcultured. The cells are dissociated by trypsinization with 0.25% trypsin and resuspended in a volume of growth medium to achieve a concentration of approximately 500,000 cells/mL. Approximately 100 μL of the cells are added to each well of a 96 well microtiter plate to achieve a final concentration of 50,000 cells per well. The plate containing the cells is incubated for approximately 24 h and then, serial dilutions of the tested extracts (same concentrations as antimalarial assay) are made and added to duplicate wells. The cells are incubated with the test samples for an additional 48 h and the viability of the cells is determined relative to control wells. Amphotericin B is used as a positive control. The neutral red assay is used to determine the viability of the cells. See, Borenfreund, E.; Babich, H.; Martin-Alguacil, N., In vitro Dev. Cell. Biol. 1986, 26, 449. The plates are read on a Bio-Tek Model 312e microplate reader.

In Vitro Evaluation of Antimycobacterial Activity. (Tuberculosis Antimicrobial Acquisition & Coordinating Facility (TAACF)). Primary evaluation of purified compounds are conducted at 6.25 μg/mL (or molar equivalent of highest molecular weight compound in a series of congeners) against Mycobacterium tuberculosis H₃₇Rv (ATCC 27294) in BACTEC 12B medium using a broth microdilution assay, the Microplate Alamar Blue Assay (MABA). Compounds exhibiting fluorescence are tested in a BACTEC 460 radiometric system. See, Collins, L.; Franzblau, S. G. “Microplate Alamar Blue Assay versus BACTEC 460 System for High-throughput Screening of Compounds against Mycobacterium tuberculosis and Mycobacterium avium”, Antimicrob. Agents Chemother. 1997, 41, 1004-9. Marine compounds (>0.7 mgs) showing <90% inhibition in the primary screen (MIC>6.25 μg/mL) will not be evaluated further. Marine natural products or semisynthetics demonstrating at least 90% inhibition in the primary screen will be reassayed at lower concentrations against M. tuberculosis H₃₇Rv to determine an actual MIC using MABA. The MIC is defined as the lowest concentration effecting a reduction in fluorescence of 90% relative to controls. Along with the determination of MICs, the compounds will be tested for cytotoxicity (IC₅₀) in VERO cells at concentration ≦62.5 μg/mL or 10^{x} the MIC for M. tuberculosis H₃₇Rv (solubility in media permitting). After 72 hours of exposure, viability is measured on the basis of cellular conversion of MTT into a formazan product using the Promega CellTiter 96 Non-radioactive Cell Proliferation Assay. Compounds for which the selectivity index (i.e., IC₅₀:MIC ratio) SI >10 will have in vitro activity confirmed in the BACTEC 460 at 6.25 μg/mL. Compounds are then tested for killing of M. tuberculosis Erdman (ATCC 35801) in monolayers of mouse bone marrow macrophages See, Skinner, P. S.; Furney, S. K.; Jacobs, M. R.; Klopman, G.; Ellner, J. J.; Orme, I. M., “A Bone Marrow-Derived Murine Macrophage Model for Evaluating Efficacy of Antimycobacterial Drugs under Relevant Physiological Conditions,” Antimicrob. Agents Chemother. 1994, 38, 2557-63. (EC₉₀ and EC₉₉; lowest concentration effecting a 90% and 99% reduction, respectively, in colony forming units at seven days compared to drug-free controls) at 4-fold concentrations equivalent to 0.25, 1, 4 and 16× the MIC. At the same time that compounds are being assayed in macrophages, MICs are determined in the MABA against a strain of M. avium (ATCC 25291) and against three strains of single-drug-resistant (SDR) M. tuberculosis. The natural products and derivatives maybe utilized against TB strains resistant to isoniazid (ATTC 35822), rifampin (ATCC 35838), and one additional SDR strain chosen on the basis of the structural class of the marine natural product; confirmatory testing also occurs against drug-sensitive M. tuberculosis H₃₇Rv and Erdman. The minimum bactericidal concentration (MBC) will be determined for M. tuberculosis H₃₇Rv and Erdman by subculturing onto drug-free solid media and enumerating colony forming units following exposure in supplemented Middlebrook 7H9 media to drug concentrations equivalent to and higher than the previously determined MICs of the respective strains. The initial MIC determination can be accomplished with as little as 0.7 mgs, with the remainder of the in vitro evaluations requiring about 10 mgs.

In vivo evaluation of Anti-M. avium Activity. (TAACF assay) The evaluation of marine derived compounds against M. avium is available for compounds showing an M. avium MIC ≦6.25 μg/mL. An expanded primary screen is conducted at a range of 1 μg/mL-64 μg/mL against five M. avium clinical isolates (100, 101, 108, 109, 116) in Middlebrook 7H9 broth using the MABA and BACTEC 460 systems. Those compounds with an MIC ≧8 μg/mL in at least three of the five strains tested are reassayed at lower concentrations against 30 strains, including five strains resistant to clarithromycin. Those marine derived compounds demonstrating significant activity against the panel are tested for intracellular activity in an infected macrophage model using the human monocyte cell line U937, against three M. avium strains (100, 101, 109) representing the three serotypes encountered in AIDS patients. If activity is seen against any of the strains, ethambutol (4 μg/mL) is added in combination with the lowest concentration of the evaluated compound that showed activity in order to examine potential synergism. For promising molecules, in vivo activity is studied in a mouse model for M. avium infection. Beige-C57BL/j bg female mice are infected i.v. with 3510⁷ cfu of bacilli. After one week, therapy is initiated and continued for four weeks. The liver and spleen are then aseptically dissected, weighed, and homogenized. Serial dilutions are plated onto 7H11 agar for quantitative culture. For the in vitro evaluation of marine derived compounds against M. avium 25-30 mgs will be required.

1. A method of treating an infectious disease, cancer/tumor, inflammatory diseases or condition in a subject in need of such treatment comprising administering to the subject an effective amount of a manzamine or manzamine analog including, manadomanzamine, papuamine, ircinal, ircinol or manzamine derivative or analog or an optical isomer or racemate or tautomer thereof or a pharmaceutically acceptable salt thereof.

2. The method according to claim 1, wherein the disease or condition is caused by a parasite.

3. The method according to claim 2, wherein the parasite is Plasmodium falciparum (Malaria), P. berghei, P. yoelli, P. chabaudi, Trypanosoma brucei (Sleeping sickness), T. gambiense, T. rhodesiense, T. cruzi (Chagas disease), T. colubriformis (Filaria), Leishmania infantum (Leishmania), or L. donovani Nematodes.

4. The method according to claim 1, wherein the disease or condition is caused by bacteria.

5. The method according to claim 4, wherein the bacteria is Enterococcus coli, E. faecalis, Bacillus subtilus, B. anthraxes, Staphylococcus aureus, S. epidermidis, Pseudomonas aeruginosa, Trichophyton mentagrophytes, Streptococcus pyogenes, Salmonella sp., Mycobacterium tuberculosis or M. intracellulare.

6. The method according to claim 1, wherein the disease or condition is a fungal infection.

7. The method according to claim 6, wherein the opportunistic infection is caused by Candida albicans, C. tropicalis, C. kephyr, Cryptococcus neoformans, Aspergillus flavus, A. fumigatus, Microsporum canis, Trichophyton rubrum, T. mentagrophytes, T. quinckeanum, Cryptosporidium spp. or Toxoplasma gondii.

8. The method according to claim 1, wherein the disease or condition is caused by a virus.

9. The method according to claim 8, wherein the virus is HIV-1, HIV-2 (Human acquired immunodeficiency virus), HIV transmission inhibition, herpes viruses (HSV-1, HSV-2, VZV, EBV, CMV, HHV-6, HHV-7, HHV-8), respiratory viruses (Flu A & B, RSV, PIV, MV, HRV, Ad), hepatitis B and C virus, orthopoxviruses (Vaccinia, Cowpox), special pathogens: VEE, Punta Toro, Pichinde, Yellow fever, West Nile, Herpes viruses (HSV-1, HSV-2, HCMVSCID-hu, MCMV, GPCMV), respiratory viruses (Flu A & B, RSV, PIV-3, MV), hepatitis viruses (WHV, HDV, HBV transgenic), orthopoxviruses (Vaccinia, Cowpox), papillomaviruses (Shope, HPVSCID-hu), vesicular stomatitis virus (BHK/VSV), or human rhinovirus (HRV).

10. The method according to claim 1, wherein the disease or condition is caused by a cancer or tumor (reference earlier patents).

11. The method according to claim 1, wherein the disease or condition is caused by an inflammatory disease or inflammation (reference earlier patents).

12. The method according to any one of claim 1 wherein the manadomanzamine, papuamine ircinol, ircinal or manzamine derivative or analog is a derivative formed at a chemically reactive positions of the manzamines scaffold, including but not limited to a reductive amination product formed at the carbonyl carbons of a manzamine a hydrogen, halogen, hydroxy, oxy, C₁-C₁₂-alkoxy, C₁-C₁₂-acyloxy, amide, lower mono or dialkyl amino, aminal, thiol, C₁-C₁₂-alkylthiol, nitro, C₁-C₁₂-alkysulfonyl, aminosulfonyl, hydroxyl sulfonyl, C₁-C₁₂-acylamino, sulphate, C₁-C₁₂-alkyl, C₁-C₁₂-acyl or aryl groups including other drugs and natural products formed at the C-1 position of ircinol or ircinal, or any combination of these modifications.

13. A pharmaceutical composition for the treatment of a cancer, inflammatory, infectious disease or condition comprising a manzamine derivative or analog or an optical isomer or racemate or tautomer thereof or a pharmaceutically acceptable salt thereof and a pharmaceutically acceptable carrier or excipient.

14. The pharmaceutical composition according to claim 13, wherein the disease or condition is caused by a parasite.

15. The pharmaceutical composition according to claim 14, wherein the parasite is Acanthamoeba castellani, Babesia microti, Cryptosporidium parvum, Entamoeba histolytica, Isospora belli, Leishmania chagasi, L. donovani, L. infantum, Naegleria fowleri, Plasmodium berghei, P. chabaudi, P. falciparum, P. malariae, P. ovale, P. vivax, P. yoelli, Pneumocystis carinii, Toxoplasma gondii, Trypanosoma brucei, T. brucei gambiense, T. brucei rhodesiense, T. cruzi, T. colubriformis, Nematodes [Intestinal (Ancylostoma duodenale, Ascaris lumbricoides, Necator americanus, Strongyloides stercoralis, Trichostrongylus spp., Trichuris trichiura)], [Tissue (Brugia malayi, Loa loa, Onchocerca volvulus, Trichinella spiralis, Wuchereria bancrofti)]

16. The pharmaceutical composition according to claim 13, wherein the disease or condition is caused by bacteria.

17. The pharmaceutical composition according to claim 16, wherein the bacteria is Bacillus anthracis, B. subtilis, Borrelia burgdorferi, B. duttoni, B. hermsii, B. parkeri, B. recurrentis, B. turicatae, Bordatella parapertussis, B. pertussis, Branhamella catarrhalis, Chlamydia pneumoniae, C. psittaci, C. trachomatis, Clostridium botulism, C. difficile, C. novyi, C. perfringens, C. septicum, C. tetani, Corynebacterium diphtheriae, C. equi, C. haemolyticum, C. minutissimum, C. pseudodiphtheriticum, C. pseudotuberculosis, C. ulcerans, C. xerosis, C. jeikeium, Coxiella burnetii, Ehrlichia sennetsu, Eikenella corrodens, Enterobacter aerogenes, E. cloacae, Enterococcus coli, E. faecalis, Escherichia coli, Flavobacterium meningosepticum, Francisella tularensis, Haemophilus aegyptius, H. ducreyi, H. influenzae, H. parainfluenzae, H. para-aphrophilus, Hafnia alvei, Klebsiella pneumoniae, K. pneumoniae ssp. aerogenes, K. pneumoniae ssp. ozaenae, K. pneumoniae ssp. pneumoniae, K. pneumoniae ssp. rhinoscleromatis, Legionella pneumophila, L. pneumophila ssp. fraseri, L. pneumophila ssp. pneumophila, Leptospira biflexa, L. interrogans, Listeria monocytogenes, Moraxella phenylpyruvica, M. catarrhalis, M. lacunata, Morganella morganii, Mycobacterium avium-intracelluare, M. intracelluare, M. leprae, M. tuberculosis, Mycoplasma pneumoniae, Neisseria gonorrhoeae, N. meningitidis, Nocardia asteroides, Pasteurella pneumotropica, P. ureae, P. multocida, Peptostreptococcus spp., Proteus mirabilis, Pseudomonas aeruginosa, P. pseudomallei, Rickettsia prowazekii, R. rickettsii, R. sibirica, R. tsutsugamushi, R. typhi, Salmonella paratyphi, S. typhi, Serratia marcescens, Spirillum minus, Staphylococcus aureus, S. saprophyticus, S. epidermis, Streptobacillus moniliformis, Streptococcus agalactiae, S. anginosus, S. bovis, S. mitior, S. mutans, S. pneumoniae, S. pyogenes, S. salivanus, S. salivarius, S. sanguis, Treponema endemicum, T. pertenue, T. pallidum, Vibrio cholerae, Yersinia enterocolitica, Y. pseudotuberculosis, Y. pestis

18. The pharmaceutical composition according to claim 13, wherein the disease or condition is a fungal infection.

19. The pharmaceutical composition according to claim 18, wherein the fungal infection is caused by Aspergillus flavus, A. fumigatus, Blastomyces dermatitidis, Candida albicans, C. glabrata, C. guilliermondii, C. lusitaniae, C. parapsilosis, C. tropicalis, C. zephyr, Coccidioides immitis, Cryptococcus neoformans, Cryptosporidium spp., Fusarium ssp., Histoplasma capsulatum, H. duboisii, Mucor spp., Paracoccidioides brasiliensis, Penicillium spp., Pseudallescheria boydii, Rhizopus spp., Trichophyton rubrum, T. mentagrophytes, T. quinckeanum, Trichosporon beigelii

20. The pharmaceutical composition according to claim 13, wherein the disease or condition is caused by a virus.

21. The pharmaceutical composition according to claim 20, wherein the virus is Arbovirus (alphavirus, flavivirus, togavirus, bunyavirus), Arenavirus (Lassa fever virus), Coronavirus (HCV, 229E & OC43; HECV), Filovirus (Marburg, Ebola virus), Hepadnavirus (HBV, HDV, HEV, HCV), Lyssavirus (Rabies virus), Herpes virus (CMV, HCMVSCID-hu, MCMV, GPCMV, EBV, HSV-1, HSV-2, HHV6, HHV7, HHV8, VZV, Herpes B), Orbivirus, Papovavirus (HPV, polyomavirus), Poxvirus (monkey pox, small pox), Parvovirus (parvovirus B-19), Pircornavirus (coxsackie virus, echovirus, entervirus 70 & 71, HAV, poliovirus), Respiratory viruses (PIV, MV, HRV, AD), Orthomyxovirus (influenza A, B, C), Paramyxovirus (measles, mumps, NDV, parainfluenza virus, respiratory syncytial virus), Orthopoxvirus (Vaccinia, Cowpox), Retrovirus (HTLV-1, HTLV-2, HIV, HIV transmission inhibition, HIV-1, HIV-2)

22. The method according to claim 11, wherein the disease or condition is caused by a cancer, tumor or any other type of neoplasm. Applications for use of the compounds are described in, for example, U.S. Pat. Nos. 4,895,852; 4,895,853; 4,895,854 and International Patent Application number PCT/US00/07974; which are herein incorporated in their entirety by reference thereto.

23. The method according to claim 11, wherein the disease or condition is caused by a neurogenic inflammation, meningitis, septic shock, Down's syndrome, postischemic brain injury, HIV encephalopathy, Parkinson's disease, Alzheimer's disease, amyotrophic lateral sclerosis and multiple sclerosis.

24. A manzamine (manadomanzamine, papuamine, ircinal, ircinol) derivative or analog which is formed by modifying an O, N, olefin, aromatic, allylic, or any other chemically reactive position of the common manzamine scaffold. These modifications include but are not limited to a reduction or reductive amination product formed at a carbonyl carbon of manzamines a hydrogen, halogen, hydroxy, oxy, C₁-C₁₂-alkoxy, C₁-C₁₂-acyloxy, amide, amino, aminal, thiol, C₁-C₁₂-alkylthiol, nitro, C₁-C₁₂-alkysulfonyl, aminosulfonyl, hydroxyl sulfonyl, C₁-C₁₂-acylamino, sulphate, C₁-C₁₂-alkyl, C₁-C₁₂-acyl or aryl groups including other drugs and natural products formed at the C-1 position of ircinol, or any combination thereof.

25. A derivative in which ircinal or ircinol is reacted to form an aromatic, aliphatic, heterocyclic, or other moiety at the aldehyde position of ircinal or hydroxyl position of ircinol, including those products in which ircinal or ircinol have been reduced at the olefinic positions or esterified or alkylated at hydroxyl and amine groups as in claim 24.
